(19) United States
(12) Patent Application Publication
(10) Pub. No.: US 2022/0119599 A1
(43) Pub. Date: Apr. 21, 2022

(54) HYBRID HYDROGEL CARRIER FOR HIGH-SALINITY WASTEWATER TREATMENT AND PREPARATION METHOD THEREOF

(71) Applicant: Tongji University, Shanghai (CN)

(72) Inventors: Yalei ZHANG, Shanghai (CN); Xuefei ZHOU, Shanghai (CN); Xiaogang YOU, Shanghai (CN); Jiabin CHEN, Shanghai (CN); Tao ZHANG, Shanghai (CN); Yinchuan YANG, Shanghai (CN)

(73) Assignee: Tongji University, Shanghai (CN)

(21) Appl. No.: 17/133,660

(22) Filed: Dec. 24, 2020

(30) Foreign Application Priority Data
Oct. 15, 2020 (CN) 202011102158.1

(51) Int. Cl. C08J 3/075; C02F 3/34; C08J 3/24; C01G 49/08

(52) U.S. Cl. CPC C08J 3/075; C02F 3/347; C02F 3/341; C02F 2101/30; C08J 3/247; C01G 49/08; C01P 2006/42; C08J 3/242

(57) ABSTRACT

A hybrid hydrogel carrier for high-salinity wastewater treatment and a preparation method thereof are disclosed. The hybrid hydrogel carrier includes a functional microorganism and a conductive hydrogel carrier, wherein the functional microorganism is a halotolerant species; the conductive hydrogel carrier is a compatible conductive hybrid hydrogel, and magnetic triiron tetraoxide (Fe₃O₄) particles and a compatible substance are uniformly distributed on the surface and inside. The preparation method includes dissolving an aniline solution and a phytic acid solution in a polyvinyl alcohol solution, and cooling the mixed solution to obtain solution I; dispersing a microbial solution, the compatible substance and the Fe₃O₄ particles into the solution I to obtain solution II; dissolving ammonium persulfate in deionized water to prepare an ammonium persulfate solution, after cooling the solution, mixing quickly with the solution II to obtain solution III, then freezing and thawing the solution III repeatedly to obtain the hybrid hydrogel carrier.

### CROSS REFERENCE TO THE RELATED APPLICATIONS

This application is based upon and claims priority to Chinese Patent Application No. 202011102158.1, filed on Oct. 15, 2020, the entire contents of which are incorporated herein by reference.

### TECHNICAL FIELD

The present invention relates to the technical field of high-salinity wastewater treatment, and in particular to a hybrid hydrogel carrier for high-salinity wastewater treatment and a preparation method thereof.

### BACKGROUND

A large amount of high-salinity wastewater is produced from many industrial processes, such as textile process, printing and dyeing, petrochemical industry, leather making, pharmaceutical production, food processing and seawater utilization. Besides having a high-concentration of inorganic ions (such as Na⁺, K⁺, Ca²⁺, SO₄²⁻ and Cl⁻), the wastewater usually contains a high-concentration of organic pollutants. If the wastewater is discharged directly without proper treatment, it will pollute the receiving water body and even endanger the ecological environment and human health. Currently, physical and chemical methods for treating high-salinity wastewater have substantial investment and operation costs and often cause secondary pollution. These methods, therefore, are not ubiquitous in the aforementioned industrial production processes. The biological method is often used because of its low processing cost, wide application range, and limited impact in causing secondary pollution.

However, inorganic salts increase the density of wastewater and make the activated sludge buoyant in solution, which inevitably leads to the loss of bacteria and the difficulty of solid-liquid separation in the treatment of high-salinity wastewater using the biological method. The immobilized microorganism technology can immobilize free organisms in limited areas through physical and chemical actions, and can purify and maintain high-efficient strain compared with the traditional suspension biological treatment. However, the traditional immobilized microorganism technology is to imbed the microbial strains directly with polyvinyl alcohol or sodium alginate, which can cause negative impacts, such as easily-broken carrier particles, large mass transfer resistance, floating of product gas and great loss of activity, etc. Moreover, in the high-salinity environment, increasing osmotic pressure will result in dehydration of microbial cells, decrease of enzyme activity of microbial cells and be toxic to microorganisms.

### SUMMARY

In view of the above-mentioned problems, the objective of the present invention is to provide a hybrid hydrogel carrier for high-salinity wastewater treatment and a preparation method thereof. The new method is capable of improving the microbial load and mass transfer performance of the immobilized carrier, and combining the soluble substances with the immobilized cell technology, thereby improving the tolerance and mass transfer performance of the microorganism in the high-salinity environment.

To achieve the above objective, the present invention provides the following technical solutions.

A hybrid hydrogel carrier for high-salinity wastewater treatment provided by the present invention, including a functional microorganism and a conductive hydrogel carrier, wherein the functional microorganism is a halotolerant species.

The conductive hydrogel carrier is a compatible conductive hybrid hydrogel, and magnetic triiron tetraoxide (Fe₃O₄) particles and a compatible substance are uniformly distributed on the surface and inside.

Preferably, the microorganism is at least one selected from the group consisting of halophilic bacteria, halotolerant bacteria and halotolerant yeast.

A preparation method of the hybrid hydrogel carrier for the high-salinity wastewater treatment, including the following steps:

step 1: dissolving an aniline solution and a phytic acid solution in a polyvinyl alcohol solution, and cooling the mixed solution to obtain solution I;

step 2: dispersing a microbial solution, the compatible substance and the Fe₃O₄ particles into the cooled solution I in step 1 to obtain solution II;

step 3: dissolving ammonium persulfate in deionized water to prepare an ammonium persulfate solution, after cooling the ammonium persulfate solution, mixing the ammonium persulfate solution quickly with the solution II to obtain solution III, and then freezing and thawing the solution III repeatedly to obtain the hybrid hydrogel carrier for the high-salinity wastewater treatment.

Preferably, the phytic acid solution in step 1 is a crosslinking agent and doping agent of the hybrid hydrogel carrier with a mass fraction of 50%, and a molar ratio of aniline and phytic acid is 2:1-7:1.

Preferably, a mass fraction of the polyvinyl alcohol solution in step 1 is 4-6%, a volume ratio of the polyvinyl alcohol solution and the phytic acid solution is 2:1, and a cooling temperature of the solution I is 4° C.

Preferably, the compatible substance in step 2 is used as an osmotic protectant for the microorganism, and includes trehalose, glutamic acid or betaine, etc., and a mass concentration of the compatible substance is 100-300 mg/L.

Preferably, a particle size of the Fe₃O₄ particles in step 2 is 50-100 nm, and a concentration is 100-300 mg/L. The treatment process is as follows: preforming an ultrasonic dispersion at 20-35° C. for 30-60 min, and a cooling temperature of the solution II in step 2 is 4° C.

Preferably, a molar concentration of the ammonium persulfate solution after being dissolved in step 3 is 1.25 mmol/L, a volume ratio of the ammonium persulfate solution and the aniline solution is 2:1, and a cooling temperature of the ammonium persulfate solution is 4° C.

Preferably, a freezing temperature of the solution III in step 3 is −20° C.

Preferably, the treatment process of the solution III is as follows: after the mixture is completely solidified, thawing (4° C.) and freezing (−20° C.) the mixture repeatedly for 3 times.

The advantages of the present invention are as follows.

1. The present invention has a good three-dimensional porous structure by immobilizing the microorganism through conductive hydrogel hybridization. Compared with the traditional biological embedding method, the present invention has better biocompatibility and can effectively immobilize more microorganisms, while the combination of microorganism and carrier is more firm, and the mechanical strength of the gel is improved.

2. Fe₃O₄ nanoparticles can be uniformly distributed inside the gel. The doping of the Fe₃O₄ nanoparticles can significantly enhance the current density of the gel, which can serve as a mediator for microbial electron transfer, enhance the interspecies interaction of microorganisms and the ability to degrade organic pollutants.

3. The compatible substance doped in the conductive hydrogel can effectively improve the salt-tolerance performance of the microorganism. In a high-salinity environment, the microorganism can absorb the compatible substance fixed in the conductive hydrogel to counteract the high osmotic pressure outside, reduce the loss of water from cells, stabilize the biological macromolecular structure, and maintain the activity of cells and intracellular enzymes.

4. The pore structure and mass transfer efficiency of the conductive hydrogel can be further improved by absorption of the compatible substance in the conductive hydrogel by cells.

### BRIEF DESCRIPTION OF THE DRAWINGS

In order to more clearly describe the embodiments of the present invention or the technical solution in the prior art, a brief description of the drawings to be used in the embodiments or in the prior art description is provided below. It is obvious that the drawings described below are only some embodiments of the present invention. For ordinary technical personnel in the art, other drawings can be obtained from these drawings without creative work.

FIG. 1 is a diagram showing a preparation process of the hybrid hydrogel carrier of the present invention.

FIG. 2 is a line chart showing change of chemical oxygen demand (COD) with time in the process of high-salinity simulated wastewater treatment by the hybrid hydrogel carrier.

### DETAILED DESCRIPTION OF THE EMBODIMENTS

In order to make the objective, technical solution and advantages of the present invention clearer, the technical solution of the present invention will be described in detail below. Obviously, the described embodiments are only part of the embodiments of the present invention, not all of them. Based on the embodiments of the present invention, all other embodiments obtained by ordinary technical personnel in the art without creative labor all fall within the protection scope of the present invention.

### Embodiment 1

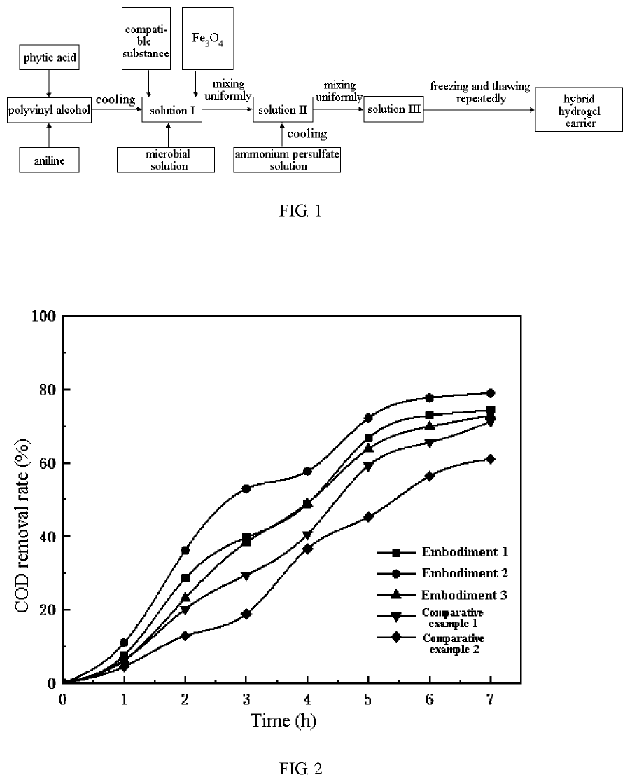

Referring to FIG. 1, a preparation method of a hybrid hydrogel carrier for high-salinity wastewater treatment includes the following steps:

step 1: phytic acid is prepared into a solution with a mass fraction of 50%, and dissolved, along with 1/5 volume of aniline, in a 4% polyvinyl alcohol solution, and then the mixed solution is cooled to 4° C. to obtain solution I;

step 2: Fe₃O₄ particles with a particle size of 50-100 nm are prepared into a solution with a concentration of 100 mg/L, and subjected to an ultrasonic dispersion at 20-35° C. for 30-60 min, then, a microbial solution containing halophilic bacteria, halotolerant bacteria and halotolerant yeast, betaine and a dispersed Fe₃O₄ solution are added into the solution I, and mixed uniformly under stirring to obtain solution II, and a mass concentration of the betaine in the solution II is 100 mg/L;

step 3: ammonium persulfate is dissolved in deionized water to prepare an ammonium persulfate solution with a molar concentration of 1.25 mmol/L (the volume is 2 times of the aniline solution), after the ammonium persulfate solution is cooled to 4° C., the ammonium persulfate solution is quickly mixed with the solution II to obtain solution III, and the solution III is transferred to −20° C. environment for freezing; when the mixture is completely solidified, the mixture is thawed (4° C.) and frozen (−20° C.) repeatedly for 3 times to obtain the hybrid hydrogel carrier for the high-salinity wastewater treatment.

### Embodiment 2

Referring to FIG. 1, a preparation method of a hybrid hydrogel carrier for high-salinity wastewater treatment includes the following steps:

step 1: phytic acid is prepared into a solution with a mass fraction of 50%, and dissolved, along with 1/2 volume of aniline, in a 4% polyvinyl alcohol solution, and then the mixed solution is cooled to 4° C. to obtain solution I;

step 2: Fe₃O₄ particles with a particle size of 50-100 nm are prepared into a solution with a concentration of 100 mg/L, and subjected to an ultrasonic dispersion at 20-35° C. for 30-60 min, then, a microbial solution containing halophilic bacteria, halotolerant bacteria and halotolerant yeast, betaine and a dispersed Fe₃O₄ solution are added into the solution I, and mixed uniformly under stirring to obtain solution II, and a mass concentration of the betaine in the solution II is 200 mg/L;

step 3: ammonium persulfate is dissolved in deionized water to prepare an ammonium persulfate solution with a molar concentration of 1.25 mmol/L (the volume is 2 times of the aniline solution), after the ammonium persulfate solution is cooled to 4° C., the ammonium persulfate solution is quickly mixed with the solution II to obtain solution III, and the solution III is transferred to −20° C. environment for freezing; when the mixture is completely solidified, the mixture is thawed (4° C.) and frozen (−20° C.) repeatedly for 3 times to obtain the hybrid hydrogel carrier for the high-salinity wastewater treatment.

### Embodiment 3

Referring to FIG. 1, a preparation method of a hybrid hydrogel carrier for high-salinity wastewater treatment includes the following steps:

step 1: phytic acid is prepared into a solution with a mass fraction of 50%, and dissolved, along with 1/2 volume of aniline, in a 4% polyvinyl alcohol solution, and then the mixed solution is cooled to 4° C. to obtain solution I;

step 2: Fe₃O₄ particles with a particle size of 50-100 nm are prepared into a solution with a concentration of 200 mg/L, and subjected to an ultrasonic dispersion at 20-35° C. for 30-60 min, then, a microbial solution containing halophilic bacteria, halotolerant bacteria and halotolerant yeast, betaine and a dispersed Fe3O4 solution are added into the solution I, and mixed uniformly under stirring to obtain solution II, and a mass concentration of the betaine in the solution II is 100 mg/L;

step 3: ammonium persulfate is dissolved in deionized water to prepare an ammonium persulfate solution with a molar concentration of 1.25 mmol/L (the volume is 2 times of the aniline solution), after the ammonium persulfate solution is cooled to 4° C., the ammonium persulfate solution is quickly mixed with the solution II to obtain solution III, and the solution III is transferred to −20° C. environment for freezing; when the mixture is completely solidified, the mixture is thawed (4° C.) and frozen (−20° C.) repeatedly for 3 times to obtain the hybrid hydrogel carrier for the high-salinity wastewater treatment.

### Comparative Example 1

Except that no Fe3O4 is added, other steps are the same as those in embodiment 2.

### Comparative Example 2

Except that no betaine is added, other steps are the same as those in embodiment 2.

The test method for applying the above-mentioned embodiments and comparative examples is as follows: the hybrid hydrogel carriers for the high-salinity wastewater treatment prepared by embodiments 1-3 and comparative examples 1-2 are put into a simulated sequencing batch reactor (SBR), respectively. The operation mode of the SBR is: inputting water for 30 min—aeration for 7 h—static sedimentation and drainage for 30 min, the drainage ratio is 50%, and the aeration volume is 10 L/min. The volume of the SBR is 2 L. The wastewater used in this test is high-salinity wastewater, which is prepared using glucose, (NH₄)₂SO₄, and K₂PO₄ according to the ratio of 100:5:1 and a certain proportion of NaCl and trace elements. The salt content of the simulated wastewater is 1000 mg/L and COD concentration is 1500 mg/L. The change of COD with time in the process of high-salinity wastewater treatment by the prepared hybrid hydrogel carrier is shown in FIG. 2.

FIG. 2 shows that the COD removal rates of the hybrid hydrogel carriers for the high-salinity wastewater treatment prepared by the embodiments 1-3 reach a maximum of 78% in 7 h, which has a significant improvement compared with the comparative examples 1-2. It indicates that the hybrid hydrogel carrier provided by the present invention for the high-salinity wastewater treatment has significant improvement effect on the salt-tolerance performance of microorganisms, and has good application prospects in the field of high-salinity wastewater treatment.

The above descriptions are only the specific implementation modes of the present invention, but the protection scope of the present invention is not limited to these. Any technical personnel familiar with the technical field can easily think of variations or replacements within the technical scope disclosed by the present invention, which shall be included in the protection scope of the present invention. Therefore, the protection scope of the present invention shall be defined by the protection scope of claims.

What is claimed is:

1. A hybrid hydrogel carrier for a high-salinity wastewater treatment, comprising a functional microorganism and a conductive hydrogel carrier,
wherein
the functional microorganism is a halotolerant species;
the conductive hydrogel carrier is a compatible conductive hybrid hydrogel, and
magnetic triiron tetraoxide (Fe₃O₄) particles and a compatible substance are uniformly distributed on a surface and an inside of the conductive hydrogel carrier.

2. The hybrid hydrogel carrier according to claim 1, wherein the functional microorganism is at least one selected from the group consisting of halophilic bacteria, halotolerant bacteria and halotolerant yeast.

3. A preparation method of the hybrid hydrogel carrier for the high-salinity wastewater treatment according to claim 1, comprising the following steps:
step 1: dissolving an aniline solution and a phytic acid solution in a polyvinyl alcohol solution to obtain a mixed solution, and cooling the mixed solution to obtain a first solution;
step 2: dispersing a microbial solution, the compatible substance and the magnetic Fe₃O₄ particles into the first solution in step 1 to obtain a second solution;
step 3: dissolving ammonium persulfate in deionized water to prepare an ammonium persulfate solution, after cooling the ammonium persulfate solution, mixing the ammonium persulfate solution quickly with the second solution to obtain a third solution, and then freezing and thawing the third solution repeatedly to obtain the hybrid hydrogel carrier for the high-salinity wastewater treatment.

4. The preparation method according to claim 3, wherein the phytic acid solution in step 1 is a crosslinking agent and a doping agent of the hybrid hydrogel carrier, a mass fraction of the phytic acid solution is 50%, and a molar ratio of aniline and phytic acid is 2:1-7:1.

5. The preparation method according to claim 3, wherein a mass fraction of the polyvinyl alcohol solution in step 1 is 4-6%, a volume ratio of the polyvinyl alcohol solution and the phytic acid solution is 2:1, and a cooling temperature of the first solution is 4° C.

6. The preparation method according to claim 3, wherein the compatible substance in step 2 is used as an osmotic protectant for the functional microorganism, the compatible substance is one selected from the group consisting of trehalose, glutamic acid and betaine, and a mass concentration of the compatible substance is 100-300 mg/L.

7. The preparation method according to claim 3, wherein a particle size of the magnetic Fe3O4 particles in step 2 is 50-100 nm, and a concentration of the magnetic Fe₃O₄ particles is 100-300 mg/L; a treatment process of step 2 is as follows: preforming an ultrasonic dispersion on the microbial solution, the compatible substance and the magnetic Fe₃O₄ particles at 20-35° C. for 30-60 min, and a cooling temperature of the second solution in step 2 is 4° C.

8. The preparation method according to claim 3, wherein a molar concentration of the ammonium persulfate solution in step 3 is 1.25 mmol/L, a volume ratio of the ammonium persulfate solution and the aniline solution is 2:1, and a cooling temperature of the ammonium persulfate solution is 4° C.

9. The preparation method according to claim 3, wherein a freezing temperature of the third solution in step 3 is −20° C.

10. The preparation method according to claim 9, wherein a treatment process of the third solution is as follows: after the third solution is completely solidified, thawing (4° C.) and freezing (−20° C.) the third solution repeatedly for 3 times.

11. The preparation method according to claim 3, wherein the functional microorganism is at least one selected from the group consisting of halophilic bacteria, halotolerant bacteria and halotolerant yeast.
